(19) United States
(12) Patent Application Publication
(10) Pub. No.: US 2016/0264492 A1
(43) Pub. Date: Sep. 15, 2016

(54) INTEGRATED PROCESS FOR CONVERTING LIGHT PARAFFINS TO GASOLINE AND DISTILLATE

(71) Applicant: ExxonMobil Research and Engineering Company, Annandale, NJ (US)

(72) Inventors: Christopher G. Smalley, Manassas, VA (US); Mohsen N. Harandi, The Woodlands, TX (US)

(21) Appl. No.: 15/060,888

(22) Filed: Mar. 4, 2016

Related U.S. Application Data
(60) Provisional application No. 62/132,079, filed on Mar. 12, 2015.

(51) Int. Cl. C07C 2/12; C07C 4/04

(52) U.S. Cl. CPC C07C 2/12; C07C 4/04; C07C 2529/40

(57) ABSTRACT

An integrated process for converting light (C₂-C₅) paraffins to hydrocarbon products in the gasoline and distillate boiling range in which a light paraffin fraction is subjected to steam cracking under conditions to maximize weight of C₅+ molecules plus a multiple of the weight of C₄− olefins. This multiple is preferably between 0.7-1 to result in maximum volume of gasoline and distillate production in the overall conversion to products. Light (C₂-C₅) olefins are oligomerized in a zeolite-catalyzed fluidized bed oligomerization at elevated temperature to produce oligomerized products in the gasoline and middle distillate boiling range.

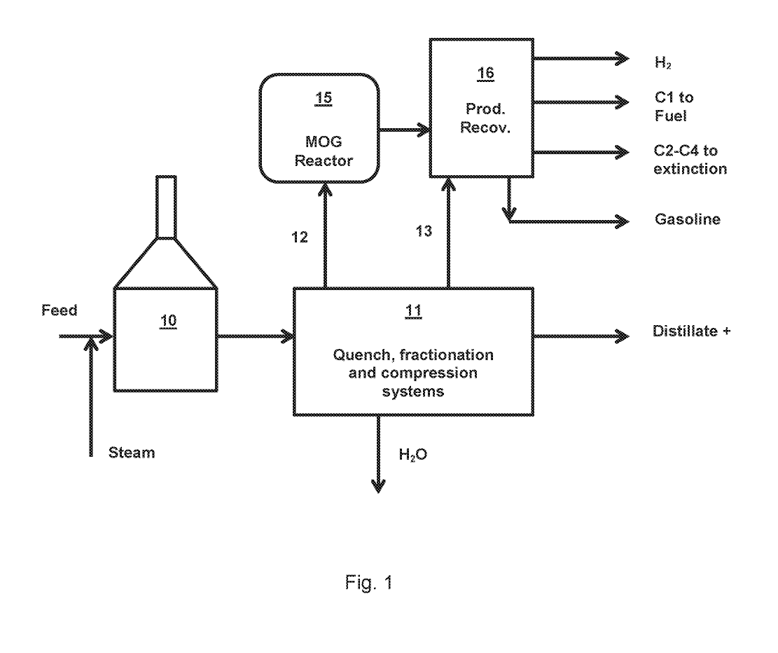

### CROSS-REFERENCE TO RELATED APPLICATIONS

This application claims priority to U.S. Provisional Application Ser. No. 62/132,079 filed Mar. 12, 2015, which is herein incorporated by reference in its entirety.

### FIELD OF THE INVENTION

This invention relates to the refining of low value hydrocarbon fractions into gasoline, distillate and higher boiling products of greater economic value at lower cost. The process is particularly applicable to the upgrading of light paraffins (C₇-C₅) which are presently in abundant supply in the United States. Light paraffins of this kind are commonly found in Natural Gas Liquids (NGL), tight oils (light crude oil contained in petroleum-bearing formations of low permeability, often shale or tight sandstone, also referred to as light tight oils), as well as fractions from various refining and/or chemical streams.

### BACKGROUND OF THE INVENTION

With the increasing production of shale gas and tight oils, the supply of light paraffins (e.g., C₂-C₈, especially C₂-C₅ paraffins) is increasing at an unprecedented rate in the North America region; a large fraction (up to 30%) of NGL, for example, is C₄/C₅ paraffins. At the same time, demand for C₄/C₅ molecules is decreasing due to a number of factors including the shrinkage of the North American gasoline pool and a potential mandate for gasoline Reid Vapor Pressure (RVP) reduction. Although diluent use of C₅s for heavy crude is predicted to grow somewhat, the supply of C₄s/C₅s is quickly outpacing demand and the imbalance is becoming worse with time. Upgrading C₄/C₅ paraffins to higher value and large volume products while desirable, remains challenging. Conversion of C₄/C₅ paraffins to heavier hydrocarbon products such as low RVP gasoline would provide a large volume and higher value outlet to help alleviate the excess of light ends in the North American market but there is no current commercial process directly converting light paraffins to heavier hydrocarbons such as low RVP gasoline.

At the same time, the crude oils which are being used for refining to liquid transportation fuels are becoming heavier and more problematical in their processing. A large proportion of the North American gasoline pool is provided by the Fluid Catalytic Cracking Process (FCC) with, at the same time, copious production of olefins and cracked cycle oil which, after hydrotreatment may be used as road diesel. Under the heavy pressure of regulation, there is a considerable incentive to reduce emissions both in the actual refining process, for example, by the elimination of nitrogen oxides and sulfur oxides from the FCC flue gas as well as from the combustion of the final refined products, for example, by major reductions in the sulfur contents of gasoline and road diesel. Removal of the most common organic contaminants (sulfur, nitrogen) is typically carried out by hydrogenative processing such as catalytic hydrodesulfurization (CHD), hydrotreating (HDT) or hydrocracking (HDC). While hydrotreatment of FCC feeds is technically attractive from the viewpoint of promoting feed crackability and removing the contaminants at an early stage in the process, it is vastly expensive in terms of hydrogen consumption and for this reason, economically unattractive. At the same time, the FCC unit itself as an item of major capital and operating expense: it is a large unit with two main. sections (cracker and regenerator) and consumes large amounts of cracking catalyst. With these considerations in mind, alternatives to the hydrogenative processing and catalytic cracking of heavy feeds are desirable.

Petroleum reefing processes in which crudes and crude fractions such as atmospheric gas oil and vacuum gas oil are converted to lower boiling products are either carbon rejection processes or hydrogen addition processes. Hydrogenative processing is, by its nature catalytic as is FCC, and both are capital and operationally expensive. Carbon rejection processes are less consumptive of capital resources and may offer reductions in operational costs by not requiring large quantities of catalyst subject to aging and deterioration. Coking, either in a delayed coker or a fluid coker, is not generally considered a highly attractive option from the viewpoint of product quality with large volumes of fairly undesirable by-product in the form of petroleum coke.

### SUMMARY OF THE INVENTION

We have now developed an integrated process which is capable of converting light paraffins in the C₂-C₅ range as well as heavier, higher boiling fractions including gas oils, crudes and topped crudes to refined products in the gasoline and distillate boiling range.

According to the present process, a light paraffin fraction, optionally with an added proportion of a high boiling (345° C.+, 650° F.+) oil, is subjected to steam cracking under relatively mild conditions to produce a product slate comprising principally light olefins (C ₂-C₅). If a heavy oil fraction is introduced. as a co-feed, distillate (440° F.+, 225° C.+) fractions increase in the product slate.

The olefin oligomerization is preferably carried out in a zeolite-catalyzed fluidized bed oligomerization process at elevated temperature to produce oligomerized products in the gasoline and. light and/or middle distillate boiling range. An integrated steam cracker/oligomerization unit may be used with a single product recovery section and with light paraffins and olefins C₂₊) from the oligomerization recycle to the cracker to participate in further reactions potentially to higher olefins which may be oligonierized to gasoline or distillate product.

### DRAWINGS

The single FIGURE of the accompanying drawings is a simplified process schematic of an integrated process unit using a single product recovery section in which a preferred form of the process in which a mild steam cracking step is used to produce light olefins for conversion in an olefin oligomerization step.

### DETAILED DESCRIPTION

### Process Configuration

In the FIGURE, the light paraffinic (C₂-C₈, typically C₂-C₅) feed is introduced with steam into steam cracker 10 in which it is cracked under relatively mild conditions. The feed may optionally include a minor proportion of heavier fractions such as crude, topped crude or gas oil, e.g. vacuum gas oil, if desired. The feed can also just be VGO, naphtha and/or distillate boiling molecules. The conditions in the cracker are set to produce a product which is predominantly light olefins (C₂-C₅); if a heavy oil co-feed is used, distillate and heavier products, e.g gas oil boiling range fractions, may also result. The conditions in the cracker are optimized to maximize weight of C₅+ molecules plus a multiple of the weight of C₄− olefins, For the reasons discussed below, this multiple is preferably between 0.7-1 to result in the maximum volume of gasoline and distillate production in the overall conversion.

The effluent from cracker 10 is passed to the quench, fractionation and compression system 11 where H₂O from the steam is removed and any distillate and heavier fractions are removed as product for further processing. The compressed, light olefinic fraction (mainly C₂-C₄ olefins with hydrogen and C₁) is taken to a Mobil Olefins-to-Gasoline (MOG) reactor 15 for oligomerization. Wild naphtha is taken directly from fractionation system 11 to cracker/MOG recovery section 16. The light olefins are oligomerized to higher boiling fractions in the MOG reactor with the effluent passing to the cracker/MOG recovery section 16 along with the wild naphtha from fractionator 11. The cracker/MOG recovery section separates the various fractions from the hydrogen which is taken to utilization. The C₁ passes to the fuel gas system and C₂-C₄ is preferably recycled through the unit to extinction. The gasoline boiling range product is removed for further processing along with any higher boiling fractions produce in the MOG reactor. The C₅+ from the MOG unit can be used for gasoline or fractionated into gasoline and distillate components. The distillate fraction should be hydrotreated to saturate olefins. Optionally the steam cracker naphtha can be upgraded and desulfurized in the MOG step.

### Steam Cracker

The feed to the steam cracker is predominantly a light paraffinic feed (C₂-C₄ with some heavier ends typically up to C₈ being normally encountered depending on the efficiency of the cut points in the preceding units. As the C₅s and higher may be profitably included in the gasoline pool, it is preferred to maximize the C₂-C₄ portion of the light paraffins. Typically, the feed to the steam cracker will comprise 80-100 vol. pct. of the light paraffins with heavy oil fractions above the naphtha (C₅-225° C./440° F.) and middle distillate (345° C.+, 650° F.+) ranges such as VGO making up the balance. Generally, the feed to the cracker will contain only a minor proportion of heavy oils, for example, not more than 10 vol. pct or less, e.g. 5, 2 or only 1 vol. pct,

The C₂C₁₀ alkane feedstock preferably comprises ethane and/or propane. Other suitable feedstocks are ethane, butane, pentane, and hexane in any of their isomeric forms, either alone or in admixture with one another.

Another useful feed is the reformer paraffins separated in an extraction unit such as Udex™. Other preferred feeds include the saturated gas plant LPG as the paraffinic feedstock and the LPG from the unsaturated gas plant with or without the ethene fraction as the olefinic feedstock. Olefinic feeds are preferably sent first to the MOG unit for upgrading; unconverted light olefins are preferably hydrogenated with the resulting paraffins then being sent to the steam cracker.

With the predominantly light paraffinic feed, the steam cracker is run under conditions which will enable the operations of the cracker and the oligomerization unit to be integrated to maximize the overall yield of the valuable gasoline and middle distillate fractions. The integration can be expressed in the following manner

The C₅+ molecules from the cracker are directly available as the desired gasoline and middle distillate product, e.g. C₅-345° C. (C₅-650° F.) product; the C₅+ fraction therefore has a weighting of I in the optimization of the two units. The C₄− olefins from the cracker are converted in the oligomerization unit where about 70 percent or more, e.g. 80 percent, 90 percent result in a product fraction in the C₅-345° C. (C₅-650° F.) range with about 30 percent of lower boiling paraffins and olefins also emerging as products of the oligomerization step. The C₄− olefins are therefore given a preferred minimum weighting of about 0.7 in the optimization. The lower boiling paraffins and olefins from the oligomerization unit can be recycled to the cracker for further reaction potentially resulting in additional C₅-345° C. (C₅-650° F.) product or C₄− olefins which can be sent back to the oligomerization unit.

Thus, the steam cracker is optimally run under conditions which maximize weight of C₅+ molecules plus a multiple of the weight of C₄− olefins. This multiple is preferably between 0.7-1 to result in maximum volume of gasoline and distillate production in the overall conversion to products. Operation of the cracker under conditions which increase the C₄− olefins beyond the 0.7 weighting factor are possible depending on the operational efficiency of the oligomerization unit and its capability to generate product in the gasoline and distillate boiling range: if, for example, the oligomerization unit can be operated with 80 or 90 percent conversion to gasoline/distillate product, a weighting factor of 0.8 or 0.9 is appropriate. Alternatively, the weighting factor may be determined by the value and utility of the C₂+ paraffin and olefin by-product at the particular location; in most cases, the weighting factor for the C₄− olefins from the cracker will, however, not exceed 1. Conditions should preferably be such as will minimize the yield of ethylene since this is less reactive to oligomerization and thus will not contribute to the objective of maximizing gasoline and distillate product. Depending on the feed composition the operating conditions can be relatively milder conditions than typical steam cracking operations for maximizing ethylene yield. The overall C₅+ gasoline and distillate yield also depends on desired RVP of gasoline product and/or how much C₅'s and heavier from steam cracker is processed in the oligomerization reactor. When a heavy oil co-feed is used, sonic aromatics and fuel oil will be included in the product slate, depending on the boiling range of the heavy oil component.

in steam cracking, the gas or liquid hydrocarbon feed is diluted with steam and heated in an oxygen-free atmosphere in a tubular furnace. The reaction temperature is very high, typically at around 850° C.; reaction severity is controlled by the brief reaction time, often measured in milliseconds. After leaving the furnace tubes, the hot effluent is quenched to stop the reaction. According to the present process, the conditions in the cracker (temperature and residence time at temperature) are controlled to make a large amount of light olefins for the oligomerization unit as described above. Preferably the C₂-C₄ olefin yield is held to at least 50 wt. pct. with butane feeds; higher yields are typically attained with the use of ethane and propane-rich feeds. Preferably, with ethane, the light (C₂-C₃) olefin yield will be at least 80 wt. pct. and at least 60 wt. pet with a propane feed. Naphtha and gas oil range feeds typically result in 40-45 wt. pet light olefins.

Typical steam cracker yields for different feeds are shown below.

| | Light Feeds | | | Heavy Feeds | |
|---|---|---|---|---|---|
| (Yield by weight) | Ethane | Propane | Butane | Naphtha | Gas Oil |
| Hydrogen & methane | 13% | 28% | 24% | 26% | 18% |
| Ethylene | 80% | 45% | 37% | 30% | 25% |
| Propylene | 2% | 15% | 18% | 13% | 14% |
| Butadiene | 1% | 2% | 2% | 5% | 5% |
| Mixed butenes | 2% | 1% | 6% | 8% | 6% |
| C₅+ | 2% | 9% | 13% | 8% | 7% |
| Benzene | 0% | 0% | 0% | 5% | 5% |
| Toluene | 0% | 0% | 0% | 4% | 3% |
| Fuel oil | 0% | 0% | 0% | 2% | 18% |

### Olefin Oligomerization

The objective of the steam cracking step is to make light olefins for the olefin oligomerization which follows. Various olefin oligomerization processes may be used with catalysts such as metal catalysts. Friedel-Crafts catalysts, etc but the preferred process is the fluidized bed, zeolite catalyzed process known as Mobil Olefins to Gasoline (MOG) process in which the vast majority of the products will be in the gasoline and distillate boiling range.

In the MOG process, the olefinic feedstream is contacted with a catalyst, normally ZSM-5, in a turbulent fluidized bed reaction zone. More than 80 wt. pct. and preferably more than 90 wt, pct. of the intermediate olefins is normally converted to the oligomer product. Operating conditions for the turbulent flow reaction zone typically comprise temperatures of 215° C. to 540° C. and pressures of about 50 to 2000 kPa and a WHSV of about 0.140. The catalyst, which preferably comprises ZSM-5, may have an acid cracking (alpha) value of about 2 to 35, preferably about 5-10. Optionally, a co-feed comprising C₂+ alkenes can be introduced into the upper turbulent oligomerization zone as a quench to maintain thermal balance in the fluid bed. The product comprising C₅+ gasoline and middle distillate range hydrocarbons and unreacted alkanes and hydrogen from the cracker is withdrawn and enters recovery zone 16 where the various effluent fractions are separated and passed on as described above.

The oligomerization catalyst can be withdrawn for oxidative regeneration zone with an oxygen-containing gas such as air. The freshly regenerated catalyst is combined with the feed.

The catalysts preferred for use in the MOG process include crystalline aluminosilicate zeolites having a silica-to-alumina ratio of at least 12, a Constraint Index of about 1 to 12 and acid cracking activity of about 160-200. Representative of suitable zeolites are ZSM-5, ZSM-11, ZSM-12, ZSM-23, and ZSM-35. The preferred catalyst is a HZSM-5 zeolite with alumina binder in the form of cylindrical extrudates. Other catalysts which may be used include a variety of medium pore (.about. 5 to 8 Ångstroms) siliceous materials, such as borosilicates, ferrosilicates and/or aluminosilicates.

The steam cracker/MOG combination can be an alternative to catalytic cracking of VGO for high gasoline yields with minimal or no hydroprocessing.

The present integrated process combination has significant benefits on the cost and number of equipment units as compared to each individual unit being built separately; including the benefits for the steam cracking unit and the fact that only one recovery section is built rather than two:

The cost of the recovery section for conventional steam cracking unit is substantially lower since olefins are converted to gasoline ahead of the recovery section. This eliminates need for the di-olefins saturation (DIOS) unit, the refrigeration unit for recovering ethylene and fractionation systems for recovering ethylene, propylene, etc.

The majority of volume of gas to be handled is reduced by the oligomerization step, so making the remaining fractionation system smaller.

The need for refrigerated storage system for products is also eliminated and finished product piping is greatly simplified.

As all the olefins produced in the steam cracking are processed over the oligomerization catalyst, and for this reason there is less incentives to run the cracking reaction at a very low pressure or very high temperature to maximize ethylene yield. This should reduce cost of the steam cracking and its energy usage.

What is claimed is:

1. A process for converting light paraffins to hydrocarbon products in the gasoline and distillate boiling range which comprises:
subjecting a light paraffin fraction to steam cracking to produce products comprising principally light olefins, and
oligomerizing the light olefins to produce oligomerized products in the gasoline and middle distillate boiling range.

2. A process according to claim 1 in which the light paraffinic fraction is subjected to steam cracking with a high (345° C.+650° F.+) gas oil.

3. A process according to claim 2 in which the feed to the steam cracking contains not more than 5 vol. pct. of the high boiling oil.

4. A process according to claim 1 in which the light paraffinic fraction comprises C₂-C₄ paraffins.

5. A process according to claim 1 in which the C₂-C₄ olefin yield from the steam cracking is at least 50 wt. pct.

6. A process according to claim 1 in which the C₂-C₄ olefin yield from the steam cracking is at least 60 wt. pct.

7. A process according to claim 1 in which the feed to the steam cracking comprises light paraffins and a high boiling oil to produce a C₂-C₄ olefin yield from the steam cracking of at least 40 wt. pct. light olefins.

8. A process according to claim 1 in which the steam cracker is operated to maximize the weight of C₅+ molecules plus a multiple in the range of 0.7 to 1 of the weight of C₄− olefins.

9. A process according to claim 1 in which the light olefin oligomerization is carried out by contacting the light olefins with a zeolite catalyst at elevated temperature.

10. A process according to claim 9 in which the light olefin oligomerization is carried out by contacting the light olefins with a zeolite catalyst in a fluidized bed at elevated temperature.

11. A process according to claim 10 in which the light olefin oligomerization is carried out by contacting the light olefins with a zeolite catalyst in a fluidized bed at elevated temperature in the range of 215 to 510° C. and a pressure of 50 to 2000 kPa at a WHSV of about 0.1-10.

12. A process according to claim 9 in which the zeolite catalyst comprises ZSM-5.

13. A process according to claim 9 in which the zeolite catalyst comprises ZSM-5 with an acid cracking (alpha) value of 2 to 35.

14. A process according to claim 1 in which the naphtha fraction from the steam cracking is passed to a common recovery section with effluent from the olefin oligomerization step.

15. A process according to claim 1 in which the feed to the steam cracking step includes a heavy gas oil fraction and middle distillate and heavier fractions are recovered from the steam cracking step.

16. A process according to claim 1 in which the feed to the steam cracking step comprises ethane and/or propane.
